(19) United States
(12) Patent Application Publication
(10) Pub. No.: US 2023/0331762 A1
(43) Pub. Date: Oct. 19, 2023

(54) CRYSTALLINE FORM OF ASPACYTARABINE

(71) Applicant: Biosight Ltd., Lod (IL)

(72) Inventors: Ramy LIDOR-HADAS, Kfar Saba (IL); Margarita SHUMILOV, Herzliya (IL); Shoshanna TESSLER, Zichron Yaakov (IL)

(73) Assignee: Biosight Ltd., Lod (IL)

(21) Appl. No.: 18/027,396

(22) PCT Filed: Sep. 20, 2021
(86) PCT No.: PCT/IL2021/051144

§ 371 Date: Mar. 21, 2023

Related U.S. Application Data
(60) Provisional application No. 63/080,808, filed on Sep. 21, 2020.

(51) Int. Cl. C07H 19/09

(52) U.S. Cl. CPC C07H 19/09; C07B 2200/13

(57) ABSTRACT

The present invention relates to a novel crystalline polymorph of (¾)-2-amino-4-(0-42R,3S,4S,5R)-3,4-dihydroxy-5-(hydroxymethyptetrahydrofuran-2-yl)-2-oxo-1,2- dihydropyrimidin-4-yl)amino)-4-oxobutanoic acid (also known as BST-236, Astarabine® or aspacytarabine), processes of preparation thereof, and uses thereof for the treatment of neoplastic diseases.

### FIELD OF THE INVENTION

The present invention relates to a novel crystalline polymorph of (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (also known as BST-236, Astarabine® or aspacytarabine), processes of preparation thereof, and uses thereof for the treatment of neoplastic diseases.

### BACKGROUND OF THE INVENTION

The use of prodrugs to impart desired characteristics such as bioavailability, pharmacokinetics, or increased site-specificity is a recognized concept in the art of pharmaceutical development. For example, direct or indirect conjugation of a drug to an antibody creates a stable conjugate that can arrive at the target site with minimum dissociation of the drug. Drug targeting may be combined with a mechanism of selective release of the drug for maximal potency.

International Patent Application Publication No. WO/2017/093993 teaches prodrugs comprising cytarabine conjugated to a single amino acid selected from the group consisting of aspartic acid, glutamic acid, asparagine, and glutamine, for use in treating neoplastic diseases in medically compromised subjects. Aspacytarabine is a conjugate of cytarabine and aspartic acid wherein cytarabine is covalently attached to the carboxyl group of the side chain of aspartic acid. It is useful for treatment neoplastic diseases including hematological cancers such as acute lymphocytic leukemia (ALL), acute myeloid leukemia (AML), or myelodysplastic syndrome (MDS), thereby prolonging the survival of the patients in need of the treatment.

Polymorphs, solvates, and salts of various drugs have been described in the literature as imparting novel properties to the drugs. Organic small drug molecules have a tendency to self-assemble into various polymorphic forms depending on the environment that drives the self assembly.

Identifying which polymorphic form is the most stable under each condition of interest and the processes that lead to changes in the polymorphic form is crucial to the design of the drug manufacturing process in order to ensure that the final product is in its preferred polymorphic form. Different polymorphic forms of an active pharmaceutical ingredient (API) can lead to changes in the drug's solubility, dissolution rate, pharmacokinetics and ultimately its bioavailability and efficacy in patients.

Solid materials can be in an amorphous form that lacks the long-range order that is characteristic of a crystal solid. Crystalline materials may have more than one form of crystal structure that differs in the arrangements or conformations of the molecules in the crystal lattice.

The crystalline forms and amorphous form of drug molecules have similar chemical structures, molecular formulas and molecular configurations, but differ in physicochemical properties like stability and solubility. Crystallization may increase the stability of amorphous drug substances. For example, amorphous penicillin G is less stable than crystalline salt and Amitriptyline is more stable in crystalline form than in amorphous form.

Disappearing polymorphs is a known phenomenon that refers to a solid form that has been prepared at least once and whose existence has been established experimentally by some observation or measurement. Subsequent attempts to prepare the same solid form by the same procedure lead to a different solid form, alone or together with the old one. If a mixture appears in the first instance, very often in subsequent preparations the new form dominates, and the old form is no longer obtained. The old—“disappeared”—form is generally less stable than the new one under those specific conditions. In thermodynamic terms, it is metastable, although that does not necessarily imply that it would spontaneously convert into a more stable form; it only means that it is at a higher energy minimum than the most stable state.

This invention provides a new thermodynamically stable polymorph of Aspacytarabine and process of preparation thereof to ensure reproducible manufacturing of Aspacytarabine.

### SUMMARY OF THE INVENTION

In one aspect, this invention relates to a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine).

In some embodiments, the crystalline polymorph of the compound is characterized by an X-Ray Powder diffraction pattern comprising unique peaks at ° 2θ±0.2 (d value Å); 16.4 (5.4), 19.8 (4.5) and 20.9 (4.3) when obtained with a Cu tube anode with K-alpha radiation. In other embodiments, the crystalline polymorph of the compound is characterized by an X-Ray Powder diffraction pattern comprising unique peaks at ° 2θ±0.2 (d value Å); 12.6 (7.0), 12.8 (6.9), 16.4 (5.4), 18.6 (4.8), 19.8 (4.5), 20.9 (4.3), 26.5 (3.4) when obtained with a Cu tube anode with K-alpha radiation. In other embodiments, the crystalline polymorph of the compound is characterized by an X-Ray Powder diffraction pattern as shown in FIG. 2 and FIG. 3.

In other embodiments, crystalline polymorph of aspacytarabine has a chemical purity of more than 95%.

In one aspect, this invention provides a composition comprising a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of the invention and a pharmaceutically acceptable carrier.

In other embodiment, the composition comprises a crystalline polymorph of aspacytarabine and an amorphous form of aspacytarabine. In one embodiment, this invention provides a process for the preparation of a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of this invention, the process comprises:
(i) removing a CBz (benzyloxycarbonyl) and a Bn (benzyl) groups of Compound 3 [benzyl N²-((benzyloxy)carbonyl)-N⁴-(1-((2R,3S,4S,5R)-3,4-dihydroxy-5-(hydroxymethyl)tetrahydrofuran-2-yl)-2-oxo-1,2-dihydropyrimidin- 4-yl)-L-asparaginate] by catalytic hydrogenation comprising Hz, a catalyst, an acidic water and an organic solvent;
(ii) followed by adjusting the pH to 2-9 and precipitation to obtain Form B.

In one embodiment, this invention provides a process for the preparation of a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of this invention, the process comprises:
(i) removing a CBz (benzyloxycarbonyl) and a Bn (benzyl) group of Compound 3 [benzyl N²-((benzyloxy)carbonyl)-N⁴-(1-((2R,3S,4S,5R)-3,4-dihydroxy-5-(hydroxymethyl)tetrahydrofuran-2-yl)-2-oxo-1,2-dihydropyrimidin-4-yl)- L-asparaginate] by catalytic hydrogenation comprising Hz, a catalyst, in an amide solvent and water mixture;
(ii) adding dichloromethane, toluene, acetonitrile, 2-Me-THF, ethyl acetate, ethanol or any combination thereof to the reaction mixture and extracting the aqueous phase;
(iii) followed by precipitation of aspacytarabine Form B with or without adding antisolvent to the aqueous phase.

In one embodiment, this invention provides a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine), which is prepared by the process of this invention.

In another aspect, the present invention provides a method of treating a neoplastic disease comprising administering to a subject in need thereof a composition comprising a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of the invention.

In some embodiments, this invention provides a process for the preparation of (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid-salt (aspacytarabine-salt), wherein the salt is prepared by reacting the crystalline polymorph (Form B) of this invention with a strong acid.

### BRIEF DESCRIPTION OF THE FIGURES

The subject matter regarded as the present invention is particularly pointed out and distinctly claimed in the concluding portion of the specification. The present invention, however, both as to organization and method of operation, together with objects, features, and advantages thereof, may best be understood by reference to the following detailed description when read with the accompanying drawings in which:

FIG. 1 depicts the XRPD pattern of amorphous aspacytarabine form.

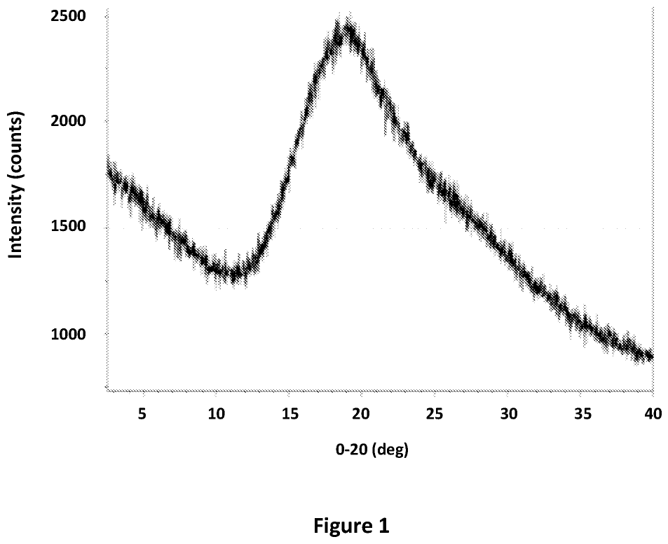

FIG. 2 depicts the XRPD pattern of aspacytarabine crystalline polymorph (Form B) measured by PANalytical X'Pert PRO MPD diffractometer and X'Celerator detector.

FIG. 3 depicts the XRPD pattern of aspacytarabine crystalline polymorph (Form B) measured by Bruker AXS D2 diffractometer and a LynxEye detector.

FIG. 4 presents a synthetic scheme of process for the preparation of aspacytarabine-HCl.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The present invention provides a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine).

In some embodiments, the crystalline polymorph (Form B) of the compound is an anhydrous crystalline form. In another embodiment, the crystalline polymorph (Form B) of the compound is a hydrate crystalline form. In another embodiment, the crystalline polymorph (Form B) of the compound is a solvate crystalline form.

In some embodiments, the crystalline polymorph of the compound is characterized by an X-Ray Powder diffraction pattern comprising unique peaks at ° 2θ±0.2 (d value Å); 16.4 (5.4), 19.8 (4.5) and 20.8 (4.3) when obtained with a Cu tube anode with K-alpha radiation.

In some embodiments, the crystalline polymorph (Form B) is characterized by an X-Ray powder diffraction pattern comprising unique peaks at ° 274 ±0.2 (d value Å); 16.4 (5.4), 18.6 (4.8), 19.8 (4.5), 20.9 (0,3), 26.5 (3.4) when obtained \Nith a Cu tube anode with K-alpha radiation.

In some embodiments, the crystalline polymorph (Form B) is characterized by an X tray powder diffraction pattern comprising unique peaks at ° 2θ±0.2 (d value Å); 12.6 (7.0), 12.8 (6.9), 16.4 (5.4), 18.6 (4.8), 19.8 (4.5), 20.9 (4.3), 24.2 (3.7), 26.5 (3.4), 28.9 (3.1), 32.5 (2.7) when obtained with a Cu tube anode with K-alpha radiation when obtained with a Cu tube anode with K-alpha radiation.

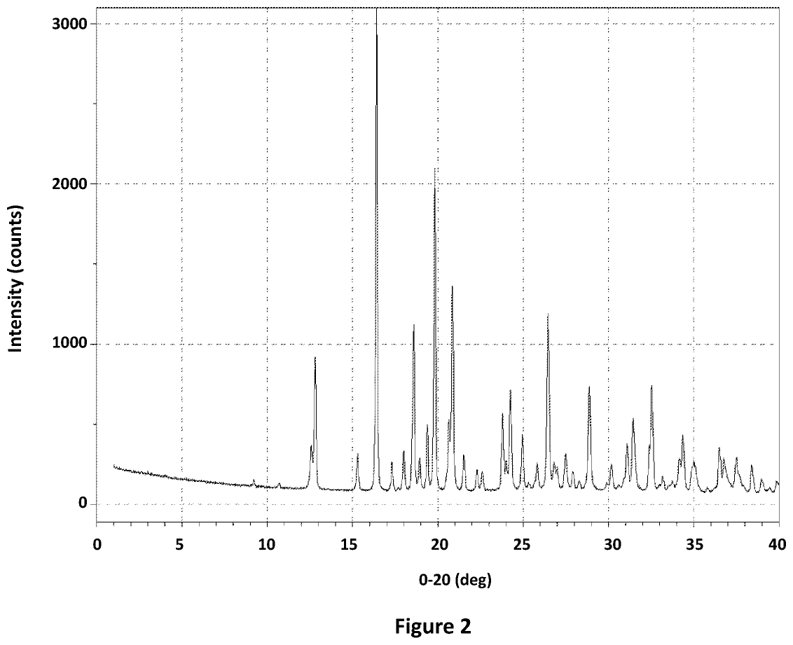

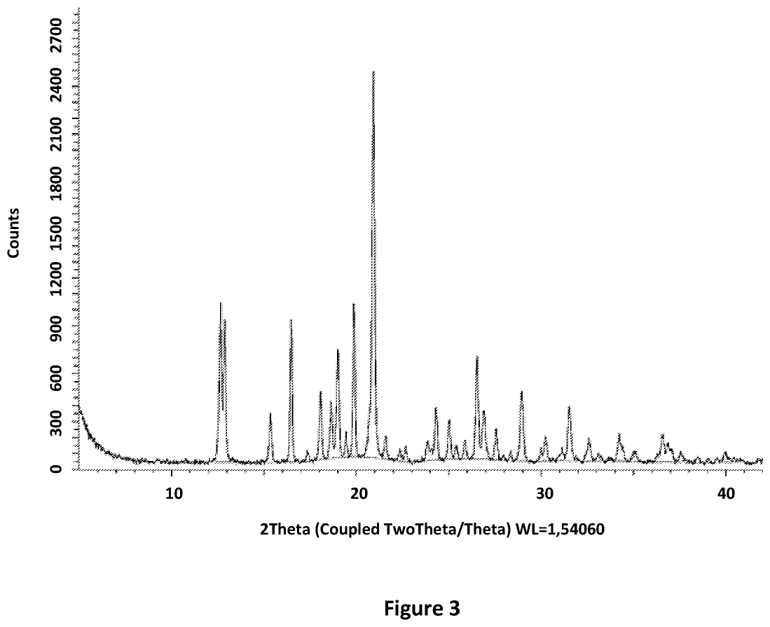

In other embodiments, the crystalline polymorph of the compound is characterized by an X-Ray Powder diffraction pattern comprising unique peaks at ° 2θ±0.2 (d value Å); 12.6 (7.0), 12.8 (6.9), 16.4 (5.4), 18.6 (4.8), 19.8 (4.5), 20.9 (4.3), 26.5 (3.4) when obtained with a Cu tube anode with K-alpha radiation. In other embodiments, the crystalline polymorph of the compound is characterized by an X-Ray Powder diffraction pattern as shown in FIG. 2. In other embodiments, the crystalline polymorph of the compound is characterized by an X-Ray Powder diffraction pattern as shown in FIG. 3.

In some embodiments, the crystalline polymorph (Form B) is characterized by an X-ray powder diffraction pattern as depicted in FIG. 2.

In some embodiments, the crystalline polymorph (Form B) is characterized by an X-ray powder diffraction pattern as depicted in FIG. 3.

In another embodiment, FIG. 2 and FIG. 3 are essentially the same (within the ° 2θ±0.2 range (d value Å); XRPD diffractogram measurements of Form B of aspacytarabine prepared from different batches and instruments.

In some embodiments, the chemical purity of the crystalline polymorph (Form B) of aspacytarabine of the invention is from about 95% to about 100%. In some embodiments, the chemical purity of the crystalline polymorph of aspacytarabine of the invention is from about 96% to about 100%. In some embodiments, the chemical purity of the crystalline polymorph of aspacytarabine of the invention is from about 97% to about 100%. In some embodiments, the chemical purity of the crystalline polymorph of aspacytarabine of the invention is from about 98% to about 100%. In some embodiments, the chemical purity of the crystalline polymorph of aspacytarabine of the invention is from about 99% to about 100%. In some embodiments, the crystalline polymorph of aspacytarabine of the invention has a chemical purity of about 99%. In some embodiments, the crystalline polymorph of aspacytarabine of the invention has a chemical purity of more than 98%. In some embodiments, the crystalline polymorph of aspacytarabine has a chemical purity of more than 95%.

In some embodiments, the crystalline polymorph (Form B) of aspacytarabine is not soluble in most organic solvents. In some embodiments, the organic solvent comprises toluene, ethyl acetate, DCM, EtOH, MeOH, and THF. In other embodiments, the organic solvent is toluene. In certain embodiments, the organic solvent is DCM. In other embodiments, the organic solvent is EtOH. In some embodiments, the organic solvent is MeOH. In other embodiments, the organic solvent is THF.

In some embodiments, the crystalline polymorph (Form B) of aspacytarabine is soluble in an amide solvent. Non limiting examples of an amide solvent include N-Methyl-2-pyrrolidone (NMP), dimethylformamide (DMF), dimethylacetamide (DMA), formamide, N-methylformamide, 2-pyrrolidone or any combination thereof. In another embodiment, the crystalline polymorph (Form B) of aspacytarabine is soluble in NMP.

In some embodiments, the crystalline polymorph (Form B) of aspacytarabine is not soluble in water. Crystalline polymorph (Form B) is a form of Aspacytarabine, not soluble in water (compared to the amorphous form which is soluble in water 100 mg/mL) and is thermodynamically more stable than the amorphous form.

In some embodiments, the crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) is used for the preparation of (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid salt thereof (aspacytarabine-salt).

In some embodiment, this invention provides a process for the preparation of (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid-salt (aspacytarabine-salt), wherein the salt is prepared by reacting the crystalline polymorph (Form B) of this invention with a strong acid.

In another embodiment the aspacytarabine-salt comprises a strong acid salt. In another embodiment the salt is selected from the group consisting of, hydrochloride salt, hydrobromide salt, TFA salt, methanesulfonate salt, phosphate salt, toluenesulfonate salt, benzenesulfonate salt, bisulfate salt and sulfate salt. In one embodiment, the salt is a hydrochloride salt. In one embodiment, the salt is a hydrobromide salt. In one embodiment, the salt is a TFA salt. Each possibility represents a separate embodiment of the invention. In another embodiment, the aspacytarabine-salt is soluble in water.

In another embodiments, aspacytarabine-HCl decompose in water (rate is determined by heat and time). In another embodiments, aspacytarabine-HCl decompose in strong polar aprotic solvents such as DMSO and DMF.

Due to this solubility character, aspacytarabine-HCl cannot be purified after its formation and, thus it should be prepared from a pure penultimate intermediate in a reaction that will be very pure as well.

### Aspacytarabine Pharmaceutical Composition

According to one aspect, the present invention provides a pharmaceutical composition comprising a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of the invention and a pharmaceutically acceptable carrier.

In some embodiment, this invention provides a pharmaceutical composition comprising a combination of crystalline polymorph (Form B) of aspacytarabine and an amorphous form of aspacytarabine. In other embodiments, the weight ratio between the crystalline polymorph and the amorphous form is in the range of between 10:1 to 1:10. In another embodiment the weight ratio between the crystalline polymorph and the amorphous form is 1:1, 2:1, 3:1, 4:1, 5:1, 6:1, 7:1, 8:1, 9:1, 10:1, 1:10, 1:9, 1:8, 1:7, 1:6, 1:5, 1:4, 1:3 or 1:2 or any ranges thereof.

According to one aspect, the present invention provides a pharmaceutical composition comprising a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of the invention and at least one stabilizer and/or solubilizer selected from a linear polymer, cyclodextrin and combination thereof.

In one embodiment, the weight ratio between the aspacytarabine and the stabilizer and/or solubilizer is between 99:1 and 1:10. In one embodiment, the ratio is between 99:1 to 99:9. In another embodiment, the ratio is between 99:9 to 99:49. In another embodiment, the ratio is between 99:49 to 1:1. In another embodiment, the ratio is between 1:2 to 1:5. In another embodiment, the ratio is between 1:5 to 1:10. Each possibility represents a separate embodiment of the present invention. In another embodiment, the weight ratio is between 80:20 and 60:40. In another embodiment, the weight ratio is between 40:60 and 20:80. In another embodiment, the weight ratio is between 30:70 and 10:90.

In another embodiment, the weight percentage of aspacytarabine is between 1% and 99%, relative to the total weight of the composition. In another embodiment, the weight percentage of aspacytarabine is between 75% and 95%, relative to the total weight of the composition. In another embodiment, the weight percentage of aspacytarabine is between 50% and 80%, relative to the total weight of the composition. In another embodiment, the weight percentage of aspacytarabine is between 10% and 80%, relative to the total weight of the composition. In another embodiment, the weight percentage of aspacytarabine is between 10% and 50%, relative to the total weight of the composition. In another embodiment, the weight percentage of aspacytarabine is between 10% and 30%, relative to the total weight of the composition. In another embodiment, the weight percentage of aspacytarabine is between 5% and 15%, relative to the total weight of the composition. In another embodiment, the weight percentage of aspacytarabine is between 1% and 10%, relative to the total weight of the composition. Each possibility represents a separate embodiment of the invention.

In one embodiment, the weight percentage of the at least one linear polymer and/or cyclodextrin is between 0.1 and 30% w/w relative to the total weight of the composition. In another embodiment, the weight percentage is between 0.1 and 0.5% w/w relative to the total weight of the composition. In another embodiment, the weight percentage is between 0.5 and 1% w/w relative to the total weight of the composition. In another embodiment, the weight percentage is between 1 and 2% w/w relative to the total weight of the composition. In another embodiment, the weight percentage is between 2 and 5% w/w relative to the total weight of the composition. In another embodiment, the weight percentage is between 5 and 10% w/w relative to the total weight of the composition. In another embodiment, the weight percentage is between 10 and 20% w/w relative to the total weight of the composition. In another embodiment, the weight percentage is 1 or 3% w/w relative to the total weight of the composition. Each possibility represents a separate embodiment of the invention.

In one embodiment, the at least one stabilizer and/or solubilizer is a water-soluble linear polymer. In another embodiment, the linear polymer is ionic or non-ionic. In some embodiments, non-ionic water soluble linear polymer comprise poly(vinyl alcohol), polyacrylamide, polyethylene glycol (polyethylene oxide) (PEG), polyethylene oxide (PEO) or polyoxyethylene (POE), triblock copolymers comprising polyoxypropylene (poly(propylene oxide)) two polyoxyethylene (poly(ethylene oxide)) (Poloxamer), polyvinyl pyrrolidone (PVP), derivative thereof or any combination thereof. In some embodiments, ionic water soluble linear polymer comprise ionic derivatives of poly(vinyl alcohol), polyacrylamide, polyethylene glycol (polyethylene oxide) (PEG), polyethylene oxide (PEO) or polyoxyethylene (POE), triblock copolymers comprising polyoxypropylene (poly(propylene oxide)) and two polyoxyethylene (p oly (ethyl ene oxide)) (Poloxamer), polyvinyl pyrrolidone (PVP), polystyrene sulfonic acid, polystyrene sulfonates derivatives thereof or any combination thereof.

In another embodiment, the at least one linear polymer is poloxamer. In another embodiment, the at least one water soluble linear polymer is combination of poloxamer and polyvinyl pyrrolidone (PVP). In another embodiment, the at least one water soluble linear polymer is cyclodextrin. In another embodiment, non-limiting examples of cyclodextrin (CD) include α-CD, β-CD, γ-CD, HP-β-CD (hydroxypropylated), SBE-β-CD (sulfobutyl-ether—modified), RM-β-CD (randomly methylated) and any combination thereof. Each possibility represents a separate embodiment of the present invention.

In another embodiment, the composition as described herein comprises aspacytarabine and poloxamer. In another embodiment, the composition comprises aspacytarabine and a combination of poloxamer and polyvinyl pyrrolidone (PVP). In another embodiment, the composition comprises aspacytarabine and cyclodextrin. Each possibility represents a separate embodiment of the present invention.

In one embodiment, the composition is chemically and physically stable when stored at a temperature of between −80 to 30° C. for at least 24 hrs as an aqueous solution or at least for one month as a dry formulation.

“Chemical stability” is herein defined as stability due to inertness of aspacytarabine compound within the composition. Thus, high chemical stability means reduced propensity of aspacytarabine compound to react/decompose in a chemical reaction over time.

“Physical stability” is herein defined as stability of a composition due to reduced possibility of changes in the physical, macro (visible) structure of the composition. Thus, “high physical stability” means for example a clear aqueous solution of the composition as described herein followed by suspension with time.

The term “water-soluble stabilizer” refers to a chemical ingredient that stabilizes the aspacytarabine or pharmaceutically acceptable salt thereof and prevents its decomposition. In some embodiments, the water-soluble stabilizer is also a solubilizer. In some embodiments, the water-soluble stabilizer is selected from a water soluble linear polymer, a cyclodextrin or combination thereof.

In one embodiment, the storage temperature range for the composition as described herein or an aqueous solution comprising thereof is between −80 to 30° C. In another embodiment, the storage temperature range for the aqueous solution is between 15-30° C. In another embodiment, the storage temperature range for the aqueous solution is between 25-30° C. In another embodiment, the storage temperature range for the aqueous solution is between 2-8° C. In another embodiment, the storage temperature range for the aqueous solution is between 8-15° C. In another embodiment, the storage temperature range for the aqueous solution is between 0-15° C. In another embodiment, the storage temperature range for the aqueous solution is between 0-10° C. In another embodiment, the storage temperature range for the aqueous solution is between 0-20° C. In another embodiment, the storage temperature range for the aqueous solution is between 0-30° C. In another embodiment, the storage temperature range for the aqueous solution is between −80 to 10° C. Each possibility represents a separate embodiment of the present invention.

In one embodiment, the composition as described herein is stable at the temperature ranges discussed above for at least 1 month. In another embodiment, the composition is stable for at least 2 months. In another embodiment, the composition is stable for at least 3 months. In another embodiment, the composition is stable for at least 4 months. In another embodiment, the composition is stable for at least 5 months. In another embodiment, the composition is stable for at least 6 months. In another embodiment, the composition is stable for at least 7 months. In another embodiment, the composition is stable for at least 8 months. In another embodiment, the composition is stable for at least 9 months. In another embodiment, the composition is stable for at least 10 months. In another embodiment, the composition is stable for at least 11 months. In another embodiment, the composition is stable for at least one year. In another embodiment, the composition is stable for between 1 and 3 months. In another embodiment, the composition is stable for between 3 and 6 months. In another embodiment, the composition is stable for between 6 and 9 months. In another embodiment, the composition is stable for between 6 and 12 months. In another embodiment, the composition is stable for between 1 and 2 years. Each possibility represents a separate embodiment of the present invention.

In one embodiment, the composition as described herein is formulated as an aqueous solution and is stable for at least 24 hrs as an aqueous solution at 0-30° C. In another embodiment, the aqueous solution is stable for between 24 hrs to 36 hrs at 0-30° C. In another embodiment, the aqueous solution is stable for between 24 hrs to 48 hrs at 0-30° C. In another embodiment, the aqueous solution is stable for between 24 hrs to 72 hrs at 0-30° C. In another embodiment, the aqueous solution is stable for between 24 hrs up to a week at 0-30° C. In another embodiment, the aqueous solution is stable for between 24 hrs to 10 days at 0-30° C.

In one embodiment, the composition as described herein is formulated as a parenteral, oral, intranasal or inhalation composition. In one embodiment, the parenteral composition is selected from a solution, a suspension, an emulsion for injection or infusion, particles for injection or infusion, liposomes as injectable delivery system, a powder for injection or infusion, and a gel for injection. In another embodiment, the parenteral composition is administered by intravenous, intraarterial, intramuscular, subcutaneous, intraperitoneal, intracerebral, intracerebroventricular, intrathecal or intradermal administration route. In another embodiment, the oral composition is selected from a tablet, a pill, a capsule, a drage, a gel, a syrup, a slurry, a suspension, a powder, or a liquid form. Each possibility represents a separate embodiment of the present invention.

In one embodiment, the composition as described herein further comprises a pharmaceutically acceptable carrier. In one embodiment, the carrier is water, saline solution, isotonic solution, aqueous dextrose, multiple electrolyte injection or aqueous glycerol solution. Each possibility represents a separate embodiment of the present invention.

In one embodiment, the composition as described herein is formulated for infusion or injection in a pharmaceutically acceptable carrier, wherein the carrier is selected from water, saline solution, isotonic solution, solutions accepted for infusion, aqueous dextrose or aqueous glycerol solution, wherein the composition having a pH range of between 2.2 and 8. In one embodiment, the pH range is between 4 and 8. In another embodiment, the pH range is between 7 and 8. In another embodiment, the pH range is between 4-5. In another embodiment, the pH is physiological. In another embodiment, a buffer is used in order to maintain and/or adjust the required pH range. In another embodiment, the buffer can be a pharmaceutically acceptable mono-ionic buffer system or a poly-ionic buffer system having an ionization pK in the range of 2.2 - 8. In another embodiment, various buffers can be used, for example, ACES (N-(acetamido)-2-aminoethansulfonic acid); Acetate; N-(2-acetamido)-2-iminodiacetic acid; BES (N,N-bis[2-hydroxyethyl]-2-aminoethansulfonic acid); Bicine (2-(Bis(2-hydroxyethyl)amino)acetic acid); Bis-Tris methane (2-[Bis(2-hydroxyethyl)amino]-2-(hydroxymethyl)propane-1,3-diol); Bis-Tris propane (1,3-bis(tris(hydroxymethynmethylamino)propane); Carbonate; Citrate; 3,3-dimethyl glutarate; DIPSO (3-[N,N-bis(2-hydroxyethyl)amino]-2-hydroxypropansulfonic acid); N-ethylmorpholine; Glycerol-2-phosphate; Glycine; Glycine-amid; HEPBS (N-(2-hydroxyethyl)piperazin-N′-4-buthanesulfonic acid); HEPES (N-(2-hydroxyethyl)piperazin-N′-2-ethanesulfonic acid); HEPPS (N-(2-hydroxyethyl)piperazin-N′-(3-propanesulfonic acid)); HEPPSO (N-(2-hydroxyethyl)piperazin-N′-(2-hydroxypropanesulfonic acid); Histidine; Hydrazine; Imidazole; Maleate; 2-methylimidazole; MES (2-(N-morpholino)ethanesulfonic acid); MOBS (4-(N-morpholino)-butansulfonic acid); MOPS (3-(N-morpholino)-propanesulfonic acid; MOPSO (3-(N-morpholino)-2-hydroypropanesulfonic acid); Oxalate; Phosphate; Piperazine; PIPES (1,4-Piperazine-diethanesulfonic acid); POPSO (Piperazine-N,N′-bis(2-hydroxypropanesulfonic acid)); Succinate; Sulfite; TAPS (3-[[1,3-dihydroxy-2-(hydroxymethyl)propan-2-yl]amino]propane-1-sulfonic acid); TAPSO (3-[[1,3-dihydroxy-2-(hydroxymethyl)propan-2-yl]amino]-2-hydroxypropane-1-sulfonic acid); Tartaric acid; TES (2-[[1,3-dihydroxy-2-(hydroxymethyl)propan-2-yl]amino]ethanesulfonic acid); THAM (Tris) (2-Amino-2-hydroxymethyl-propane-1,3-diol); and Tricine (N-(2-Hydroxy-1,1-bis(hydroxymethyl)ethyl)glycine); a sulfonic acid derivative buffer including, but not limited to, ACES, BES, DIPSO, HEPBS, HEPES, HEPPS, HEPPSO, MES, MOBS, MOPS, MOPSO, PIPES, POPSO, Sulfite, TAPS, TAPSO, and TES buffer; a carboxylic acid derivative buffer including, but not limited to, Acetatate, N-(2-acetamido)-2-iminodiacetic acid, 2-(Bis(2-hydroxyethyl)amino)acetic acid, Carbonate, Citrate, 3,3-dimethyl glutarate, Lactate, Maleate, Oxalate, Succinate, and Tartaric acid buffer; an amino acid derivative buffer including, but not limited to, Bicine, Glycine, Glycine-amid, Histidine, and Tricine buffer; a phosphoric acid derivative buffer including, but not limited to, Glycerol-2-phosphate and phosphate buffer; and other buffer systems such as: Hank's balanced salt solution, Earle's balanced salt solution, Gey's balanced salt solution, HEPES buffered saline, phosphate buffered saline, Plasma-lyte, Ringer's solution, Ringer Acetate, Ringer lactate, Saline citrate, Tris buffered saline, acid-citrate-dextrose solution and Elliott's B solution; and any combination thereof. Each possibility represents a separate embodiment of the present invention.

In other embodiments, pharmaceutical compositions for parenteral administration include aqueous solutions of the active ingredients in water-soluble form. Additionally, suspensions of the active compound may be prepared as appropriate oily injection suspensions. Suitable lipophilic solvents or vehicles include fatty oils such as sesame oil, or synthetic fatty acids esters such as ethyl oleate, triglycerides or liposomes.

In other embodiment, the composition as described herein may be formulated as a liquid formulation.

Without wishing to be bound to any theory or mechanism of action, aspacytarabine, an amino-acid-cytarabine conjugate of the composition as described herein, is transported into the cancer cells and within the cells these conjugates are cleaved to release cytarabine which arrests cell growth or kill the cell. As free cytarabine and free cytarabine metabolites were detected in cancer cells, the conjugates of the present invention act as pro-drugs. These pro-drugs are stabilized and/or dissolved due to the at least one water soluble linear polymer, cyclodextrin or combination thereof employed herein in the compositions of the present invention.

The compositions can be formulated as solutions, suspensions, emulsion, tablets, pills, capsules, powders, sustained-release formulations and the like. The composition can be formulated as a suppository, with traditional binders and carriers such as triglycerides, microcrystalline cellulose, gum tragacanth or gelatin. Each possibility represents a separate embodiment of the present invention.

The composition can further comprise excipients including, but not limited to, sodium chloride, potassium chloride, magnesium chloride, sodium gluconate, sodium acetate, calcium chloride, sodium lactate, and the like. The composition, if desired, can also contain minor amounts of sugar alcohols, wetting or emulsifying agents, and pH adjusting agents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. Each possibility represents a separate embodiment of the present invention.

For oral administration, the composition as described herein can be formulated readily by combining aspacytarabine and at least one stabilizer and/or solubilizer selected from a linear polymer, cyclodextrin or combination thereof with additional components as known in the art. Such components enable the composition as described herein to be formulated as tablets, pills, dragees, capsules, liquids, gels, syrups, slurries, suspensions, and the like, for oral ingestion by a subject. Pharmacological preparations for oral use can be made using a solid component, optionally grinding the resulting mixture, and processing the mixture of granules, after adding suitable auxiliaries if desired, to obtain tablets or dragee cores. Suitable components are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose. If desired, disintegrating agents may be added, such as cross-linked polyvinyl pyrrolidone, agar or alginic acid or a salt thereof such as sodium alginate.

In addition, enteric coating can be useful if it is desirable to prevent exposure of the compounds of the invention to the gastric environment.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added.

For buccal administration, the compositions may take the form of tablets or lozenges formulated in conventional manner.

For administration by inhalation, the active compound for use according to the present invention are conveniently delivered in the form of an aerosol spray presentation from a pressurized pack or a nebulizer with the use of a suitable propellant, e. g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e. g., gelatin for use in an inhaler or insufflator may be formulated containing a powder mix of the peptide and a suitable powder base such as lactose or starch.

An intranasal composition may be formulated as a powder, an aqueous solution or a non-aqueous solution. A preferred method of administering the solutions of the invention is using a spray device. Spray devices can be single (“unit”) dose or multiple dose systems. The powder formulation is preferably administered to the patient in aerosolized form whereby energy from patient inhalation (sniffing) is used to aerosolize the powder into the nasal cavity or where the device itself provides the aerosolization energy, such as via compressed air.

### Process of Preparing Aspacytarabine Solid Form

In one embodiment, aspacytarabine precipitates from an aqueous solution, from acidic aqueous solution, from organic solvent or from mixtures thereof. In another embodiment the precipitate is an aspacytarabine hydrate. In another embodiment the precipitate is an anhydrous aspacytarabine. In another embodiment, the precipitate is an aspacytarabine solvate.

In one embodiment, aspacytarabine precipitates from an organic solvent. In another embodiment, the organic solvent is water miscible solvent. In another embodiment, the organic solvent is water immiscible solvent. In another embodiment the precipitate is an aspacytarabine solvate. In another embodiment, the precipitate is aspacytarabine. In another embodiments, the organic solvent includes alkanes such as hexane, heptane, aromatics such as toluene or benzene, ethers such as diethyl ether and methyl tert butyl ether, halogenated solvents such as chloroform, and tetrachloromethane, esters like ethyl acetate and isopropyl acetate, ketones like acetone, methyl ethyl ketone and methyl isobutyl ketone, alcohols like methanol, ethanol, isopropanol and n-butanol, amides like dimethylformamide and N-methyl pyrrolidine and carboxylic acids like acetic acid and propionic acid. In one embodiment, aspacytarabine precipitates from a mixture of an aqueous solution and an organic solvent.

In one embodiment, the aspacytarabine precipitate is crystalline.

In one embodiment, the aspacytarabine precipitate as crystalline polymorph Form B. In one embodiment, aspacytarabine precipitate as crystalline polymorph Form B from an aqueous solution. In one embodiment, aspacytarabine precipitate as crystalline polymorph Form B from water. In one embodiment, aspacytarabine precipitate as crystalline polymorph Form B from an aqueous solution where the pH is in the range that forms a zwitterion between the NH₂ and COOH groups of aspacytarabine. In one embodiment, aspacytarabine precipitate as crystalline polymorph Form B from an aqueous solution, wherein the pH is between 2-9. In another embodiment, the pH is between 3-5. In another embodiment, the pH is between 4-7. In another embodiment, the pH is 5-8. In another embodiment, the pH is 2, 3, 4, 5, 6, 7, 8 or 9 or any ranges thereof.

In one embodiment, this invention provides a process for the preparation of a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of this invention, the process comprises crystallization from an aqueous solution, from organic solvent or from mixtures thereof. In another embodiment, the aqueous solution is in a pH range which forms a zwitterion between the NH₂ and COOH of the aspacytarabine compound.

In one embodiment, this invention provides a process for the preparation of aspacytarabine polymorph Form B, the process comprises:
(i) deprotecting any protecting group of functional groups of aspacytarabine;
(ii) following the deprotection step, aspacytarbine is crystallized to obtain Form B.

In another embodiment, the protecting groups of aspacytarabine comprise: an amino protecting group (on the aspartic alpha amino), a COOH protecting group (on the aspartic alpha carboxylic) and/or an hydroxy protecting group (hydroxy group on the arabinose sugar moiety).

In some embodiments, the deprotecting step of the amino and COOH protecting groups is conducted under H₂, in an organic solvent or an aqueous solution or mixture thereof,

In another embodiment, the deprotecting step is conducted under H₂ in a mixture of an organic solvent and an acidic aqueous phase.

In another embodiment, the deprotecting step is conducted in a mixture of an organic solvent and an aqueous phase.

In some embodiments, following the deprotection step, the pH of the aqueous phase is adjusted to pH range which forms a zwitterion, and the aspacytarabine is crystallized to obtain Form B.

In another embodiment, the pH which form a zwitterion between the NH₂ and COOH of the aspacytarabine compound is a pH between 2-9. In another embodiment, the pH is 1, 1.5, 2, 2.5, 3, 3.5, 4, 4.5, 5, 5.5, 6, 6.5, 7, 7.5, 8, 8.5, 9 or any range between each of them.

In one embodiment, this invention provides a process for the preparation of aspacytarabine polymorph Form B, the process comprises:
(i) deprotecting the protected hydroxy group/s on the arabinose sugar moiety;
(ii) removing a CBz (benzyloxycarbonyl) and a Bn (benzyl) groups by catalytic hydrogenation;
(iii) crystalizing the aspacytarbine to obtain Form B.

In one embodiment, this invention provides a process for the preparation of aspacytarabine polymorph Form B, the process comprises:
(i) removing the CBz (benzyloxycarbonyl) and the Bn (benzyl) groups of Compound 3 (benzyl N²-((benzyloxy)carbonyl)-N⁴-(1-((2R,3S,4S,5R)-3,4-dihydroxy-5-(hydroxymethyl)tetrahydrofuran-2-yl)-2-oxo-1,2-dihydropyrimidin-4-yl)-L-asparaginate) by catalytic hydrogenation comprising H₂, a catalyst, an acidic water and an organic solvent;
(ii) followed by adjusting the pH to 2-9 and precipitation to obtain Form B.

In another embodiment by adjusting the pH a zwitterion is formed resulting in the precipitation of Form B.

In another embodiment, the organic solvent is selected from methanol, ethanol, ethyl acetate or any combination thereof. In another embodiment, the catalyst of the catalytic hydrogenation is heavy metal catalyst.

In another embodiment, the heavy metal catalyst is selected from palladium (Pd), nickel (Ni), cobalt (Co), platinum (Pt), ruthenium (Ru), rhodium (Rh) or any known catalyst in the art, which are on a support. In another embodiment, the supports for the catalyst of the catalytic hydrogenation are selected from activated carbon, alumina, barium sulfate or calcium carbonate.

In another embodiment, the catalyst of the catalytic hydrogenation is palladium catalyst on carbon. In another embodiment, palladium catalyst comprises 10% palladium on carbon, 5% palladium on carbon or 5% palladium on alumina.

In another embodiment, the catalyst of the catalytic hydrogenation comprises any known catalyst in the art and include those described in detail in the book “Handbook of Heterogeneous Catalytic Hydrogenation for Organic Synthesis” by Shigeo Nishimura, Wiley, 2001, which is incorporated herein by reference.

In one embodiment, this invention provides a process for the preparation of aspacytarabine polymorph Form B, the process comprises:

(i) removing the CBz (benzyloxycarbonyl) and the Bn (benzyl) groups of Compound 3 the benzyl N²-((benzyloxy)carbonyl)-N⁴-(1-((2R,3S,4S,5R)-3,4-dihydroxy-5-(hydroxymethyl)tetrahydrofuran-2-yl)-2-oxo-1,2-dihydropyrimidin-4-yl)- L-asparaginate by catalytic hydrogenation comprising Hz, a catalyst, in an amide solvent and water mixture;
(i) adding dichloromethane, toluene, acetonitrile, 2-Me-THF, ethyl acetate, ethanol or any combination thereof to the reaction mixture and extracting the aqueous phase;
(ii) followed by precipitation of aspacytarabine Form B with or without adding antisolvent to the aqueous phase.

In another embodiment, the antisolvent is selected from acetone, acetonitrile, toluene or combination thereof. In another embodiment, the an amide solvent of the catalytic hydrogenation include N-Methyl-2-pyrrolidone (NMP), dimethylformamide (DMF), dimethylacetamide (DMA), formamide, N-methylformamide, 2-pyrrolidone or any combination thereof. In another embodiment, t the an amide solvent of the catalytic hydrogenation is NMP.

In another embodiment, removing the CBz (benzyloxycarbonyl) and the Bn (benzyl) groups of Compound 3-benzyl N²-((benzyloxy)carbonyl)-N⁴-(1-((2R,3S,4S,5R)-3,4-dihydroxy-5-(hydroxymethyl)tetrahydrofuran-2-yl)-2-oxo-1,2-dihydropyrimidin-4-yl)-L-asparaginate by catalytic hydrogenation comprising Hz, a catalyst, in N-methyl-2-pyrrolidone (NMP)/water mixture.

In another embodiment, the catalyst of catalytic hydrogenation is heavy metal catalyst.

In another embodiment, the heavy metal catalyst is selected from palladium (Pd), nickel (Ni), cobalt (Co), platinum (Pt), ruthenium (Ru), rhodium (Rh) or any known catalyst in the art, which are on a support. In another embodiment, the support for the catalyst of the catalytic hydrogenation are selected from activated carbon, alumina, barium sulfate or calcium carbonate).

In another embodiment, the catalyst of the catalytic hydrogenation is palladium catalyst on carbon. In another embodiment, palladium catalyst comprises 10% palladium on carbon, 5% palladium on carbon, 5% Pd on alumina.

In another embodiment, the catalyst of the catalytic hydrogenation comprises any known catalyst in the art and include those described in the book “Handbook of Heterogeneous Catalytic Hydrogenation for Organic Synthesis” by Shigeo Nishimura, Wiley, 2001, which is incorporated herein by reference.

In some embodiments, this invention provides a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine), which is prepared by the process of this invention.

The crystalline polymorph (Form B) of aspacytarabine in this invention is insoluble (up to 2% solubility in water) and is crystallizes during final step of the synthesis. The amorphous (Form A) of aspacytarabine is soluble in water, however it is not thermodynamically stable and crystalizes to Form B. Surprisingly, the aspacytarabine polymorph Form B is non soluble in water and also not in most organic solvents.

In one embodiment, the crystalline aspacytarabine polymorph B obtained by the process described in this invention possess very high purity (above 95%).

### Therapeutic Use

In one aspect, the present invention provides a method of treating a neoplastic disease comprising administering to a subject in need thereof a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) of the invention.

In one aspect, the present invention provides a method of treating a neoplastic disease comprising administering to a subject in need thereof an aspacytarabine acid salt, prepared from the crystalline polymorph (Form B) of compound aspacytarabine of the invention. In another embodiment, the salt is a strong acid salt. In another embodiment, the salt is a HCl salt.

According to some embodiments of the present invention, the neoplastic disease is selected from the group consisting of hematological cancers and non-hematological cancers. In another embodiment, the hematological cancer is selected from the group consisting of leukemias, lymphomas, myelomas and Myelodysplastic Syndromes (MDS).

The term “Myelodysplastic Syndromes” (MDS) refers to a heterogeneous group of hematopoietic malignancies characterized by blood cytopenias, ineffective hematopoiesis and a hypercellular bone marrow. The MDSs are preleukemic conditions in which transformation into acute myeloid leukemia (AML) occurs in approximately 30-40% of cases. Unless allogenic stem cell transplantation can be offered, MDS is generally considered to be an uncurable condition.

According to some embodiments, the MDS is selected from MDS with multilineage dysplasia (MDS-MLD), MDS with single lineage dysplasia (MDS-SLD), MDS with ring sideroblasts (MDS-RS), MDS with excess blasts (MDS-EB), MDS with isolated del(5q) and MDS unclassifiable (MDS-U). Each possibility represents a separate embodiment of the invention.

In one embodiment, leukemia is selected from the group consisting of Acute Myeloid Leukemia (AML), Acute Lymphoblastic Leukemia (ALL), Chronic Myeloid Leukemia (CIVIL), and Chronic Lymphoblastic Leukemia (CLL). In another embodiment, the AML is selected from the group consisting of newly diagnosed AML, secondary AML, and relapsed/refractory AML. In another embodiment, the lymphoma is selected from the group consisting of Hodgkin's lymphoma and non-Hodgkin's lymphoma. Each possibility represents a separate embodiment of the present invention.

Non-hematological cancers are malignant neoplasm that arises from a site other than the bone marrow and lymphoid tissue. Are not limited to, sarcoma, carcinoma, fibrosarcoma, myxosarcoma, liposarcoma, chondrosarcoma, osteogenic sarcoma, chordoma, angiosarcoma, endotheliosarcoma, mesothelioma, Ewing's tumor leiomydsarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor cervical cancer, testicular tumor, lung carcinoma, small cell lung carcinoma, bladder carcinoma, epithelial carcinoma, astrocytoma, Kaposi's sarcoma, and melanoma. Each possibility represents a separate embodiment of the invention.

Non-hematological cancers include cancers of organs, wherein the cancer of an organ includes, but is not limited to, breast cancer, bladder cancer, colon cancer, rectal cancer, endometrial cancer, kidney cancer, lung cancer, cervical cancer, pancreatic cancer, prostate cancer, testicular cancer, thyroid cancer, ovarian cancer, brain cancer including ependymoma, glioma, glioblastoma, medulloblastoma, craneopharyngioma, pinealoma, acustic neuroma, hemangioblastoma, oligodendroglioma, menangioma, neuroblastoma, retinoblastoma, and their metastasis. Each possibility represents a separate embodiment of the invention.The present invention further provides a method for the treatment of an infection caused by a viral agent that is a cancer-causing virus. Thus, the invention provides a method for the treatment of a viral infection caused by a viral oncogene. Non-limiting examples of such viruses include human papillomavirus, Hepatitis B, Hepatitis C, Epstein-Barr virus, Human T-lymphotropic virus, Kaposi's sarcoma-associated herpesvirus, and Merkel cell polyomavirus. Each possibility represents a separate embodiment of the invention.

According to some embodiments of the present invention, the crystalline polymorph of aspacytarabine of the invention is administered parenterally, orally or by inhalation. In one embodiment, the crystalline polymorph of aspacytarabine as described herein is administered by intravenous (i.v.), intraarterial, intramuscular, subcutaneous, intraperitoneal (i.p.), intracerebral, intracerebroventricular, intrathecal or intradermal administration route. In another embodiment, the crystalline polymorph of aspacytarabine as described herein is administered at a daily dose wherein the aspacytarabine dosage is ranging from about 0.3 g/m²to about 10 g/m² of the subject's surface area, for a period of at least 3 days. In another embodiment, the dosage is ranging from about 0.3 g/m² to about 1 g/m². In another embodiment, the dosage is ranging from about 1 g/m² to about 2 g/m². In another embodiment, the dosage is ranging from about 2 g/m² to about 5 g/m². In another embodiment, the dosage is ranging from about 5 g/m² to about 10 g/m². In another embodiment, the dosage is ranging from about 0.3 g/m² to about 1 g/m². In another embodiment, the period is of at least 4 days. In another embodiment, the period is of at least 5 days. In another embodiment, the period is of at least 6 days. In another embodiment, the period is of at least 7 days. In another embodiment, the period is of at least 10 days. In another embodiment, the crystalline polymorph of aspacytarabine as described herein is administered by intravenous infusion for a period ranging from 15 minutes to 24 hours. In another embodiment, the crystalline polymorph of aspacytarabine as described herein is processed to enable administered by intravenous infusion for a period ranging from 30 minutes to 24 hours. In another embodiment, the crystalline polymorph of aspacytarabine of the invention is administered by intravenous infusion for a period ranging from 15 minutes to 0.5 hours. In another embodiment, the crystalline polymorph of aspacytarabine of the invention is administered by intravenous infusion for a period ranging from 0.5 hour to 1 hour. In another embodiment, the crystalline polymorph of aspacytarabine of the invention is administered by intravenous infusion for a period ranging from 1 hour to 3 hours. Each possibility represents a separate embodiment of the present invention. The crystalline polymorph of aspacytarabine of the invention may be administered locally and may further comprise an additional active agent and/or excipient.

According to further embodiments, the crystalline polymorph of aspacytarabine of the invention is administered in a daily dosage of at least 2, 3, 5, 10, 15, 20, 30 or at least 40 times greater than the standard of care dose of cytarabine. Each possibility represents a separate embodiment of the invention.

According to some embodiments, the crystalline polymorph of aspacytarabine of the invention is administered at least once a month. According to additional embodiments, the crystalline polymorph of aspacytarabine of the invention is administered at least twice a month. According to further embodiments, the crystalline polymorph of aspacytarabine of the invention is administered at least once a week. According to yet further embodiments, the crystalline polymorph of aspacytarabine of the invention is administered at least twice a week. According to still further embodiments, the crystalline polymorph of aspacytarabine of the invention is administered once a day for at least one week. According to further embodiments, the crystalline polymorph of aspacytarabine is administered at least once a day for at least one week or until the subject reaches a remission.

According to some embodiments, the crystalline polymorph of aspacytarabine of the invention is administered once a day for at least 2, 3, 4, 5, 6, 8, 10, 12, or at least 14 consecutive days once a month. Alternatively, the crystalline polymorph of aspacytarabine of the invention is administered once a day for at least 2, 3, 4, 5, 6, or 12 days, or further alternatively the crystalline polymorph of aspacytarabine of the invention is administered every day or twice a week until the patient reaches a remission.

The crystalline polymorph of aspacytarabine of the invention can also be delivered by slow-release delivery systems, pumps, and other known delivery systems for continuous infusion. Dosing regimens may be varied to provide the desired circulating levels of a particular compound based on its pharmacokinetics. Thus, doses are calculated so that the desired circulating level of a therapeutic agent is maintained.

Typically, the effective dose is determined by the activity and efficacy of the compound and the condition of the subject as well as the body weight or surface area of the subject to be treated. The dose and the dosing regimen are also determined by the existence, nature, and extent of any adverse side effects that accompany the administration of the compounds in a particular subject.

### Definitions

As used herein a “pharmaceutical composition” refers to a preparation of one or more of the compounds described herein, or physiologically acceptable salts or solvents thereof, with other chemical components such as physiologically suitable carriers and excipients. The purpose of a pharmaceutical composition is to facilitate administration of a compound to a subject.

The terms “renal dysfunction”, “hepatic dysfunction”, “pancreatic dysfunction”, “bone marrow dysfunction” and “cerebellar dysfunction” refer to a state in which the organ/tissue function, e.g., kidney, liver, pancreas, bone marrow, and cerebellum, is decreased relative to a normal state. In general, organ/tissue dysfunction is a state characterized in that any one or more measurement values of inspection items for organ function are deviated from the range of normal values (reference values).

The terms “standard of care dose” and “the recommended maximal dose” of cytarabine are used herein interchangeably and refer to the dosage, e.g., the daily dose, of cytarabine approved by the U.S. FDA for administration to a human subject, which dosage does not cause unacceptable adverse effects and is dependent on the subject's age and physical condition so that a fit subject of 70 or less years of age can be typically treated with a daily dose of cytarabine of up to 3 g/m² (Standard dose is between 100 to 400 mg/m²) a subject of 75 or more years of age can be treated with a daily dose of cytarabine of up to 20 mg/m² of the subject's surface area. However, it should be noted that most of the subjects of 75 or more years of age cannot be treated with cytarabine at all due to its severe adverse effects.

The terms “treatment”, “treat”, “treating” and the like, are meant to include slowing, arresting or reversing the progression of a disease. These terms also include alleviating, ameliorating, attenuating, eliminating, or reducing one or more symptoms of a disease, even if the disease is not actually eliminated and even if progression of the disease is not itself slowed or reversed. A subject refers to a mammal, preferably a human being.

The term “about” in reference to a numerical value stated herein is to be understood as the stated value +/−10%.

The term “pharmaceutically acceptable salt” of a drug refers to a salt according to IUPAC conventions. Pharmaceutically acceptable salt is an inactive ingredient in a salt form combined with a drug. Typical pharmaceutically acceptable salts include those salts prepared by reaction of the compounds of the present invention with a pharmaceutically acceptable mineral, base, acid or salt. Acid salts are also known as acid addition salts (see herein below). Pharmaceutically acceptable salts are known in the art (Stahl and Wermuth, 2011, Handbook of pharmaceutical salts, Second edition). The acid is a strong acid and is selected from the group consisting of acetic acid, hydrochloric acid, hydrobromide acid, methanesulfonic acid, phosphoric acid, toluenesulfonic acid, methanesulfonic acid, benzenesulfonic acid, sulfuric acid, bisulfuric acid, and trifluoroacetic acid. In one embodiment, the salt is a hydrochloride salt. Each possibility represents a separate embodiment of the invention.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents.

### Protecting Groups

Precursors of aspacytarabine can include one or more protecting groups, an amino protecting group (on the aspartic alpha amino), a COOH protecting group (on the aspartic alpha carboxylic) and/or an hydroxy protecting group (hydroxy group on the arabinose sugar moiety).

The term “protecting group” refers to chemical residues used to block reactive sites during chemical synthesis, that enable chemical reaction to be carried out selectively at one reaction site in a multifunctional compound, other reactive sites must be temporarily blocked. The residues used to block these reactive sites called protecting groups.

The protecting group can be a hydroxyl protecting group, an amino protecting group, a carboxy protecting group, etc. As used herein, the term “OH protecting group” or “hydroxy protecting group” refers to a readily cleavable group bonded to hydroxyl groups.

According to one embodiment, the protecting group is selected from group of acetamidomethyl (Acm), acetyl (Ac), acetonide, adamantyloxy (AdaO), alfa-allyl (OAll), Alloc, benzoyl (Bz), benzyl (Bzl or Bn), benzyloxy (BzlO), benzyloxycarbonyl (Z), benzyloxymethyl (Bom), bis-dimethylamino (NIVIe2), 2-bromobenzyloxycarbonyl (2-Br-Z), t-butoxy (tBuO), t-butoxycarbonyl (Boc), t-butoxymethyl (Bum), t-butyl (tBu), t-butylthio (tButhio), 2-chlorobenzyloxycarbonyl (2-Cl-Z), 2-chlorotrityl (2-Cl-Trt), cyclohexyloxy (cHxO), 1-cyclopropyl-1-methyl-ethyl (Dmcp), 2,6-dichlorobenzyl, 4,4′-dimethoxybenzhydryl (Mbh), 1-(4,4-dimethyl-2,6-dioxo-cyclohexylidene)-3-methylbutyl (ivDde), 4{N-[1-(4,4-dimethyl-2,6-dioxo-cyclohexylidene)-3-methylbutyl]-amino} benzyloxy (ODmab), 2,4-dinitrophenyl (Dnp), fluorenylmethoxycarbonyl (Fmoc), formyl (For), Mesitylene-2-sulfonyl (Mts), 4-methoxybenzyl, 4-methoxy-2,3,6-trimethylbenzenesulfonyl (Mtr), 4-methoxytrityl (Mmt), 4-methybenzyl (MeBzl), 3-methylpent-3-yl (Mpe), 1-methyl-1-phenyl-ethyl (PhiPr), Methyl, 4-methyltrityl (Mtt), 3-nitro-2-pyridinesulfenyl (Npys), 2,2,4,6,7-pentamethyl-dihydrobenzofurane-5-sulfonyl (Pbf), 2,2,5,6,8-pentamethyl-chromane-6-sulfonyl (Pmc), tosyl (Tos), trifluoroacetyl (Tfa), trimethylacetamidomethyl (Tacm), methyl, ethyl, t-butyl, p-methoxybenzyl (PMB), 9-fluorenylmethyl (Fm), trimethylsilyl (TMS), t-butyldimethylsilyl (TBDMS), diphenylmethyl (benzhydryl, DPM), dimethoxytrityl (DMT) and the like; Triphenylmethyl (trityl,Trt) and Xanthyl (Xan), benzyloxycarbonyl, acetyl, phenylcarbonyl, or a silyl group, which can be substituted with alkyl (trialkylsilyl), with an aryl (triarylsilyl) or a combination thereof (e.g., dialkylphenylsilyl), e.g., trimethylsilyl (TMS) or t-butyldimethyl silyl (TBDMS). Each possibility represents a separate embodiment of the present invention.

A non-limiting example of a hydroxyl protecting group is an acyl group (COR wherein R=alkyl, aryl, etc.). A currently preferred acyl group is an acetyl group (i.e., OR′=acetate, OAc). Another example of a hydroxy protecting group is a silyl group, which can be substituted with alkyl (trialkylsilyl), with an aryl (triarylsilyl) or a combination thereof (e.g., dialkylphenylsilyl). A preferred example of a silyl protecting group is trimethylsilyl (TMS) or di-t-butyldimethyl silyl (TBDMS), Triisopropylsilyl (TIPS), Triethylsilyl (TES). Other examples of hydroxy protecting groups include, for example, C₁-C₄ alkyl (e.g., methyl, ethyl, propyl, butyl and the like), allyl (All), —CH₂Ph (benzyl or bzl), —CO-(C₁-C₆ alkyl), —SO₂-(C₁-C₆ alkyl), —SO₂-aryl,—COp13 Ar in which

Ar is an aryl group as defined above, and —CO-(C₁-C₆ alkyl)Ar (e.g., a carboxybenzyl (Bz) group). Other examples of hydroxy protecting groups include acid sensitive protecting groups such as tetrahydropyranyl (THP), methoxymethyl (MOM), triphenylmethyl (Trityl) and dimethoxy trityl (DMT). Each possibility represents a separate embodiment of the present invention.

In one embodiment, the term “amino protecting group” refers to a readily cleavable group bonded to amino groups.

In another embodiment, the term “amino protecting group” refers to protecting groups that are sensitive to H₂. A non-limiting example of an amino protecting group sensitive to H₂ include CBZ, p-Methoxybenzyl carbonyl (Moz or MeOZ), Benzyl (Bn), p-Methoxybenzyl (PMB) and 3,4-Dimethoxybenzyl (DMPM),

In some embodiments, the term “COOH protecting group” or “carboxy protecting group” refers to a readily cleavable group bonded to carboxy groups. In another embodiment, the term “COOH protecting group” or “carboxy protecting group” refers to protecting groups that are sensitive to H₂. A non-limiting example of a carboxy protecting group sensitive to H₂ include Bn, benzyloxymethyl ester (BOM), tetrahydropyranyl ester (THP)), triphenylmethyl ester (Tr), 9-anthrylmethyl ester, 2-(9,10-dioxo)anthrylmethyl ester, piperonyl ester and trimethylsilyl ester (TMS).

In some embodiment, the term protected cytarabine refers to at least one of Compound a, b, c and/or d:

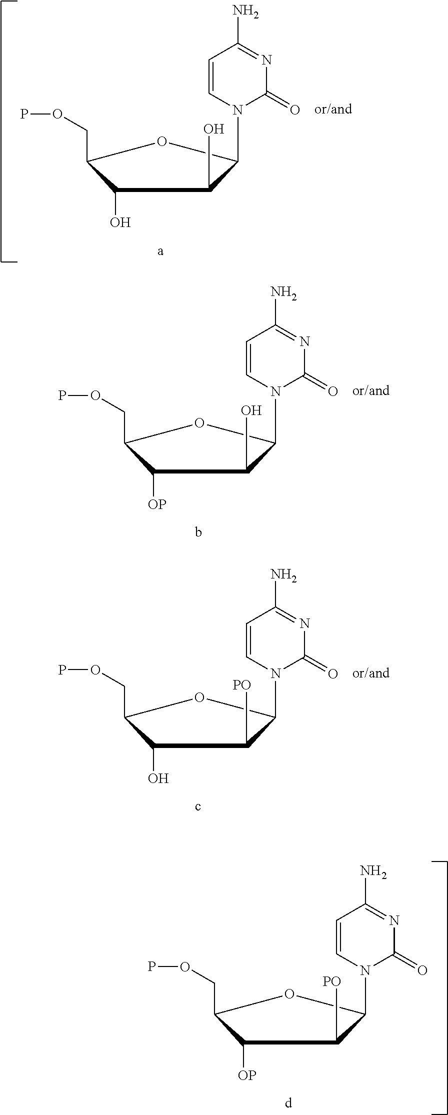

wherein P is an hydroxyl protecting group.

The following examples are to be considered merely as illustrative and non-limiting in nature. It will be apparent to one skilled in the art to which the present invention pertains that many modifications, permutations, and variations may be made without departing from the scope of the invention.

### EXAMPLES

### Example 1

Preparation of Aspacytarabine form B polymorph

Aspacytarabine crystalline polymorph Form B structure is represented by the following structure:

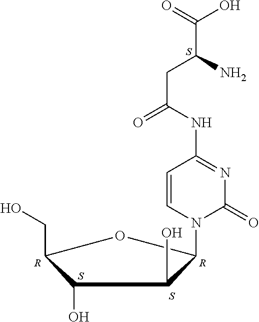

A non-limiting example for the preparation of Aspacytarabine polymorph Form B includes:

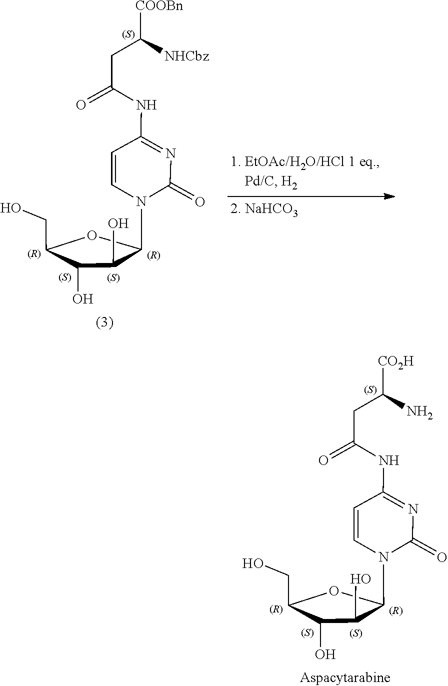

An autoclave equipped with an overhead stirrer was charged with EtOAc (700 ml, 7 vol), aqueous HCl solution (264 ml, 2.6 vol) and 10% Pd/C (1.5 g). The batch was pressure purged with nitrogen followed by hydrogen (20 psi), then stirred at 25° C. for 1 h. Bnz-Cbz-BST- 236 (Compound 3≥((benzyl N²-((benzyloxy)carbonyl)-N⁴-(1-((2R,3S,4S,5R)-3,4-dihydroxy-5-(hydroxymethyl)tetrahydrofuran-2-yl)-2-oxo-1,2-dihydropyrimidin-4-yl)-L-asparaginate) (100 g, 0.17 mol) was charged and the reactor was purged with nitrogen followed by hydrogen, then agitated under hydrogen (20 psi) at 25° C. for 2 h. The hydrogen pressure was released, solid Pd/C was filtered over celite and the reactor was rinsed with deionized water (100 ml, 1 vol). The aqueous layer was filtered through a 2-micron inline filter into a 1L jacketed reactor precooled to 2° C. The batch temperature was adjusted to 5° C. before 1N NaHCO₃ (200-210 ml) was added to the reactor at 5° C. until the batch pH reached 4.9-5.1. Heavy precipitation was observed following overnight stir. The precipitate was filtered and rinsed with water (200 ml, 2 vol) and dried under vacuum at 35° C. for 24 hours to give crystalline Aspacytarabine (Form B, 40 g, 65% yield, 98% purity).

### Example 2

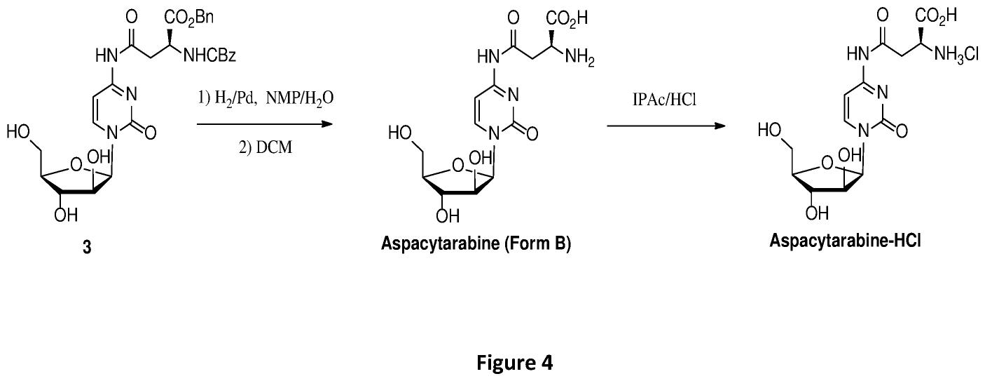

Preparation of Aspacytarabine (Form B) and Aspacytarabine HCl from Compound 3 as shown in FIG. 4

To a 2 L autoclave equipped with an overhead stirrer was charged Bnz-Cbz-BST-236 (Compound 3—((benzyl N²-((benzyloxy)carbonyl)-N⁴-(1-((2R,3S,4S,5R)-3,4-dihydroxy-5-(hydroxymethyl)tetrahydrofuran-2-yl)-2-oxo-1,2-dihydropyrimidin-4-yl)-L-asparaginate) (74.0 g, 127 mmol), Pd/C (1.11 g, 1.5% w/w) and a mixed solvent of NMP/water (740 ml/740 ml, 20 vol). The batch was pressure purged with hydrogen and stirred at 25° C. at 20 psi hydrogen for 4 hours. Solid Pd/C was filtered over celite and the reactor was rinsed to the filter with deionized water (74 ml, 1 vol). The batch was transferred to a 3 L jacketed reactor with overhead stirring followed by 7 extractions with 740 ml dichloromethane. Acetone (240 ml, 3.2 vol) was charged and the suspension formed was stirred at 25° C. for 18 hours, filtered, rinsed with acetone (600 mL) and conditioned under nitrogen for 30 minutes. The reaction batch was dried in a vacuum oven to give crystalline aspacytarabine (Form B, a white solid, 42 g, 91% yield, 99% purity).

The dried aspacytarabine was slurried with 20V of isopropyl acetate (IPAc)/HCl (2 eq) for 12 hr at 20° C. The resulting aspacytarabine-HCl salt was then filtered, washed twice with 1V IPAc, slurried again with 20V IPAc, filtered, washed twice with 1V IPAc and dried to give 99% yield of aspacytarabine-HCl.

The process provided herein provides a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) in a good yield. In another embodiment, the process provided herein provides a crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine) in at least 95% purity.

The process provided herein provides aspacytarabine from Compound 3 by catalytic hydrogenation served as intermediate in a clean solid-solid transformation process to get aspacytarabine-HCl from pure aspacytarabine free base.

### Example 3

### XRPD Indexing of Aspacytarabine crystalline Polymorph (Form B)

FIG. 2 provides XRPD diffractogram of aspacytarabine crystalline polymorph prepared according to Example 1. X-Ray Powder Diffraction patterns were collected on collected with a PANalytical X'Pert PRO MPD diffractometer using an incident beam of Cu radiation produced using an Optix long, fine-focus source. Diffraction patterns were collected using a scanning position-sensitive detector (X'Celerator) located 240 mm from the specimen.

Crystalline aspacytarabine polymorph (Form B) found to be insoluble in the most of organic solvents. This compound was not soluble in toluene, ethyl acetate, DCM, EtOH, MeOH, THF. Crystalline polymorph of aspacytarabine is not soluble in water (up to 2% in water).

The following Table provided the main XRPD peaks for Aspacytarabine Polymorph, Form B.

| Angle/ | d Value/ | |
|---|---|---|
| 2-Theta ° | Angstrom | Intensity (%) |
| 12.81 ± 0.20 | 6.905 ± 0.107 | 30 |
| 16.41 ± 0.20 | 5.397 ± 0.065 | 100 |
| 18.59 ± 0.20 | 4.769 ± 0.051 | 36 |
| 19.82 ± 0.20 | 4.476 ± 0.045 | 68 |
| 20.85 ± 0.20 | 4.257 ± 0.040 | 44 |
| 24.25 ± 0.20 | 3.667 ± 0.030 | 23 |
| 26.46 ± 0.20 | 3.366 ± 0.025 | 38 |
| 28.88 ± 0.20 | 3.089 ± 0.021 | 24 |
| 32.54 ± 0.20 | 2.749 ± 0.016 | 24 |

### Example 4

### XRPD Indexing of Aspacytarabine Crystalline Polymorph (Form B)

FIG. 3 provides XRPD diffractogram of aspacytarabine crystalline polymorph, prepared according to Example 1. X-Ray Powder Diffraction patterns were collected on a Bruker AXS D2 diffractometer using Cu K□ radiation (30 kV, 10 mA), 0-0 (reflection) geometry, using a LynxEye detector from 5-42 2θ. The details of the data collection are: Angular range: 5 to 42 ° 2θ; Step size: 0.024° 20; Collection time: 0.1 seconds per step.

The following Table provided the main XRPD peaks for Aspacytarabine Polymorph, Form B.

| Angle/ | d Value/ | | |
|---|---|---|---|
| 2-Theta ° | Angstrom | Net Intensity | Intensity (%) |
| 12.649 | 6.99260 | 667.54 | 38.9% |
| 12.879 | 6.86834 | 621.88 | 36.3% |
| 15.362 | 5.76337 | 211.91 | 12.4% |
| 16.474 | 5.37651 | 604.53 | 35.3% |
| 18.077 | 4.90320 | 300.55 | 17.5% |
| 18.644 | 4.75542 | 248.41 | 14.5% |
| 18.998 | 4.66751 | 453.26 | 26.4% |
| 19.876 | 4.46339 | 699.39 | 40.8% |
| 20.922 | 4.24259 | 1714.21 | 100.0% |
| 24.308 | 3.65876 | 240.80 | 14.0% |
| 26.532 | 3.35684 | 462.51 | 27.0% |
| 26.885 | 3.31359 | 197.60 | 11.5% |
| 28.947 | 3.08202 | 323.20 | 18.9% |
| 31.514 | 2.83656 | 248.95 | 14.5% |

The XRPD pattern indicates that Form B composed of a crystalline material. It has an orthorhombic unit cell containing four molecules of aspacytarabine. The volume of the unit cell, calculated from the indexing solution, indicates a very dense packing arrangement.

The XRPD pattern of FIGS. 2 and 3 are essentially the same as they were measured by a different machines. In FIG. 2 the XRPD pattern was measured by a PANalytical X'Pert PRO MPD diffractometer, and in FIG. 3 the XRPD pattern was measured by a Bruker AXS D2 diffractometer.

It is appreciated by persons skilled in the art that the present invention is not limited by what has been particularly shown and described hereinabove. Rather the scope of the present invention includes both combinations and sub-combinations of various features described hereinabove as well as variations and modifications. Therefore, the invention is not to be constructed as restricted to the particularly described embodiments, and the scope and concept of the invention will be more readily understood by references to the claims, which follow.

1. A crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine).

2. The crystalline polymorph of claim 1, wherein said crystalline polymorph is an anhydrous or hydrate or solvate crystalline form.

3. The crystalline polymorph of claim 1, wherein said crystalline polymorph is characterized by an X-Ray Powder diffraction pattern comprising unique peaks at ° 2θ±0.2 (d value Å); 16.4 (5.4), 19.8 (4.5) and 20.9 (4.3) when obtained with a Cu tube anode with K-alpha radiation.

4. The crystalline polymorph of claim 1, wherein said crystalline polymorph is characterized by an X-Ray Powder diffraction pattern comprising unique peaks at ° 2θ±0.2 (d value Å); 12.6 (7.0), 12.8 (6.9), 16.4 (5,4), 18,6 (4.8), 19.8 (4,5), 20,9 (4.3), 26.5 (3.4) when obtained with a Cu tube anode with K-alpha radiation.

5. The crystalline polymorph of claim 1, wherein said crystalline polymorph is characterized by an x-ray diffraction pattern as depicted in FIGS. 2 and 3.

6. The crystalline polymorph of claim 1, wherein said crystalline polymorph of aspacytarabine has a chemical purity of more than 95%.

7. A composition comprising a crystalline polymorph of compound (S)-2-amino-4-((1-((2R,3S ,4S,5R)-3,4-dihydroxy-5-(hydroxymethyl)tetrahydrofuran-2-yl)-2-oxo-1,2-dihydropyrimidin-4-yl)amino)-4-oxobutanoic acid (aspacytarabine) of claim 1 and a pharmaceutically acceptable carrier.

8. The composition of claim 7, wherein the composition comprises a crystalline polymorph of aspacytarabine and an amorphous form of aspacytarabine, wherein the weight ratio between the crystalline polymorph and the amorphous form is in the range of between 10:1 to 1:10.

9. (canceled)

10. A process for the preparation of aspacytarabine polymorph Form B of claim 1, the process comprises:
(i) removing a CBz (benzyloxycarbonyl) and a Bn (benzyl) groups of Compound 3 (benzyl N²-((benzyloxy)carbonyl)-N⁴-(14(2R,3S ,4S,5R)-3,4-dihydroxy-5-(hydroxymethyl)tetrahydrofuran-2-yl)-2-oxo-1,2-dihydropyrimidin-4-yl)- L-asparaginate) by catalytic hydrogenation comprising H2, a catalyst, an acidic water and an organic solvent;
(ii) followed by adjusting the pH to 2-9 and precipitation to obtain Form B.

11. The process of claim 10, wherein the organic solvent is selected from methanol, ethanol, ethyl acetate, or any combination thereof.

12. A process for the preparation of aspacytarabine polymorph Form B of claim 1, the process comprises:
(i) removing a CBz (benzyloxycarbonyl) and a Bn (benzyl) group of Compound 3 [benzyl N²-((benzyloxy)carbonyl)-N⁴-(1-((2R,3S ,4S,5R)-3,4-dihydroxy-5-(hydroxymethyl)tetrahydrofuran-2-yl)-2-oxo-1,2-dihydropyrimidin-4- yl)-L-asparaginate] by catalytic hydrogenation comprising H₂, a catalyst, in an amide solvent and water mixture;
(ii) adding dichloromethane, toluene, acetonitrile, 2-Me-THF, ethyl acetate, ethanol or any combination thereof to the reaction mixture and extracting the aqueous phase;
(iii) followed by precipitation of aspacytarabine Form B with or without adding antisolvent to the aqueous phase.

13. The process of claim 12, wherein the amide solvent is selected from N-Methyl-2-pyrrolidone (NMP), dimethylformamide (DMF), dimethylacetamide (DMA), formamide, N-methylformamide, 2-pyrrolidone or any combination thereof.

14. (canceled)

15. A crystalline polymorph (Form B) of compound (S)-2-amino-4-((1-((2R,3S,4S ,5R)-3,4-dihydroxy-5-(hydroxymethyl)tetrahydrofuran-2-yl)-2-oxo-1,2-dihydropyrimidin-4-yl)amino)-4-oxobutanoic acid (aspacytarabine), which is prepared by the process according to any one of claims 10.

16. A process for the preparation of (S)-2-amino-4-((1-((2R,3S ,4S,5R)-3,4-dihydroxy-5-(hydroxymethyl)tetrahydrofuran-2-yl)-2-oxo-1,2-dihydropyrimidin-4-yl)amino)-4-oxobutanoic acid-salt (aspacytarabine-salt), wherein the salt is prepared by reacting the crystalline polymorph (Form B) of claim 1 with a strong acid.

17. A method of treating a neoplastic disease comprising administering to a subject in need thereof a crystalline polymorph of aspacytarabine of claim 1.

18. The method of claim 17, wherein the neoplastic disease is selected from the group consisting of hematological cancers and non-hematological cancers, wherein said hematological cancer is selected from the group consisting of leukemias, lymphomas, myelomas and Myelodysplastic Syndromes (MDS), wherein said leukemia is selected from the group consisting of Acute Myeloid Leukemia (AML), Acute Lymphoblastic Leukemia (ALL), Chronic Myeloid Leukemia (CML), and Chronic Lymphoblastic Leukemia (CLL), wherein said AML is selected from the group consisting of newly diagnosed AML, secondary AML, and relapsed/refractory AML, and wherein said lymphoma is selected from the group consisting of Hodgkin's lymphoma and non-Hodgkin's lymphoma.

19. (canceled)

20. (canceled)

21. (canceled)

22. (canceled)

23. The method of claim 1, wherein the crystalline polymorph of aspacytarabine is administered parenterally, orally or by inhalation.

24. The method of claim 23, wherein the crystalline polymorph of aspacytarabine is administered by intravenous, intraarterial, intramuscular, subcutaneous, intraperitoneal, intracerebral, intracerebroventricular, intrathecal or intradermal administration route.

25. The method of claim 17, wherein the crystalline polymorph of aspacytarabine is administered at a daily dose wherein the aspacytarabine dosage is ranging from about 0.3 g/m² to about 10 g/m² of the subject's surface area, for a period of at least 3 days.

26. (canceled)

27. A crystalline polymorph (Form B) of compound (S)-2-amino-4-((1-((2R,3S,4S,5R)-3,4-dihydroxy-5-(hydroxymethyl)tetrahydrofuran-2-yl)-2-oxo-1,2-dihydropyrimidin-4-yl)amino)-4- oxobutanoic acid (aspacytarabine), which is prepared by the process according to any one of claims 12.
